(19) United States
(12) Patent Application Publication
(10) Pub. No.: US 2002/0172328 A1
(43) Pub. Date: Nov. 21, 2002

(54) 3-D Navigation for X-ray imaging system

(76) Inventor: Doron Dekel, Toronto (CA)

Correspondence Address: SHUMAKER & SIEFFERT, P. A., 8425 SEASONS PARKWAY, SUITE 105, ST. PAUL, MN, 55125, US

(21) Appl. No.: 10/139,003

(22) Filed: May 2, 2002

(30) Foreign Application Priority Data
May 17, 2001 (CA) 2,348,135

(51) Int. Cl. A61B 6/08

(52) U.S. Cl. 378/205

(57) ABSTRACT

A method for transforming the spatial coordinates of an instrument into its corresponding X-ray projection image is provided. The method is based on the registration of the coordinates system of an X-ray beam imaging system with a location device by simultaneously recording the spatial coordinates and the X-ray projection images of a calibration tool. The data are then used to establish a transform that will be used to convert the spatial coordinates of an instrument into its corresponding X-ray image projection with the X-ray beam turned off. Furthermore, the image of the instrument can be simultaneously displayed with that of an object to be operated on to enable an operator to guide the instrument within the object.

The present invention relates to the field of location and navigation of probes. More particularly it relates to the location and navigation of surgical instruments. Still more specifically, the invention relates to image guided surgery using X-ray fluoroscopy.

### BACKGROUND OF THE INVENTION

Surgical procedures often require that the surgeon navigate surgical instruments without being able to directly visualize their precise location within the body. To overcome this limitation, several types of surgical procedures are performed under X-ray fluoroscopic guidance. During these procedures, the physician must guide the catheter through blood vessels and organs which may have very complicated three dimensional topologies. Under fluoroscopic guidance the physician can obtain “real time” images of the catheter or of other instruments. However, these images are 2-dimensional projections and provide no information on the three dimensional position of the instrument. This, of course, limits the precision of the procedures and often leads to errors. In addition, fluoroscopes generate ionizing radiation which is harmful both for the patient and the physician. To protect themselves, medical staff wear heavy lead aprons for extended periods, often leading to back pain and other joint problems. However, fluoroscopes have the advantage that their images contain information relating the probe or catheter location to the patient's anatomy. They are also very familiar to the physician, have low operating costs and can image multiple probes and catheters simultaneously.

Three-dimensional navigation systems are available and have good accuracy relative to their own internal coordinate systems (see, for example, U.S. Pat. No. 6,104,944 to Martinelli) but cannot relate the probe, tool or catheter position relative to the patient's anatomy as displayed on the fluoroscope. The displays generated by these navigation systems show probe, tool or catheter locations relative to other probes, tools or catheters or to previous positions of these objects, but do not show any of the patient's anatomy. Consequently, the physician is required to perform procedures without anatomical guidance and to learn and understand complex and unfamiliar displays.

Methods have been devised in which probes, tools or catheters positions are registered into pre-acquired computed tomography (CT) or magnetic resonance imaging (MRI) images. The registration involves placing the probes, tools or catheters on anatomical features while simultaneously identifying the same features on the CT or MRI images. These methods are described in U.S. Pat. Nos. 5,848,967 to Cosman and 5,274,551 to Corby for example. In U.S. Pat. No. 5,274,551 a three-dimensional “map” of the anatomy is obtained with diagnostic imaging devices such as CT and MR scanners as well as X-rays and stored for later retrieval. An image of a catheter is then obtained and projected in the 3-D image of the organ or vascular network previously stored. This approach generates three-dimensional images relative to the patient's anatomy but is a complex and very expensive process. Furthermore, because of the time required to obtain CT and MRI images, diagnostic images must be obtained prior to therapy and are assumed to be accurate at the time of therapy. During the set up procedure, the patient must be meticulously registered to the image by location of fiducial points. Any anatomical movement during the operation renders the technique invalid as the anatomical imaging data cannot be refreshed during the procedure.

In view of the above limitations, there is a need for an improved method to navigate and localize the position of tools, probes and catheters in three-dimension relative to a patient's anatomy using conventional fluoroscopy.

### SUMMARY OF THE INVENTION

The present invention relates to the field of location and navigation of probes. More particularly it relates to the location and navigation of surgical instruments. Still more specifically, the invention relates to image guided surgery using X-ray fluoroscopy.

According to the present invention there is provided a method for generating and displaying an X-ray projection image of an instrument located within an object with the X-ray beam switched off. The method comprises the steps of: registering the spatial coordinates of an X-ray beam imaging system, comprising an X-ray source and an X-ray sensitive screen, with a location device; recording the spatial coordinates of the instrument using the location device with the X-ray beam switched off; transforming the spatial coordinates of the instrument in the frame of reference of the location device into the coordinates of the corresponding X-ray projection image; displaying the X-ray projection image of the instrument simultaneously with a previously stored X-ray projection image of the object.

In a further aspect of the method, registering of the coordinates system of the X-ray beam imaging system with the location device comprises the steps of: obtaining, on the X-ray sensitive screen, at least three X-ray projections of a calibration tool, the calibration tool being displaced to a new non-colinear location within the X-ray beam before each projection; determining, for each projection, the spatial coordinates of said calibration tool relative to the X-ray screen and X-ray source; recording, for each projection, the spatial location of the calibration tool relative to the X-ray sensitive screen and the X-ray source with the location device; digitizing, for each projection, the pixels intensity and location on the X-ray sensitive screen and storing them in a computer; establishing a transform to convert the spatial location of the instrument in the frame of reference of the location device into the corresponding coordinates of the corresponding X-ray projection image.

In a further aspect of the invention, the calibration tool is the same as the instrument. In this particular case, a location device is integrated within the instrument and the spatial coordinates of the instrument are registered relative to fiducial points.

In yet a further aspect of the instant invention, the image of the instrument is displayed on the display screen simultaneously with the image of an object to be operated on to visualize the position of the instrument within the object.

In another embodiment of the invention there is provided a method for generating and displaying an X-ray projection image of an instrument located within the heart while simultaneously displaying the X-ray projection image of the heart.

### BRIEF DESCRIPTION OF THE DRAWINGS

These and other features of the invention will become more apparent from the following description in which reference is made to the appended drawings wherein:

FIG. 1 is a flow chart diagram of the method of the instant invention;

FIG. 2 is a schematic diagram of a navigating system according to the instant invention;

FIG. 3 is a schematic diagram of an X-ray imaging system that can be used for the application of the instant invention, and

FIG. 4 is a schematic diagram of a navigation system in which the location device is integrated in the instrument.

### DESCRIPTION OF PREFERRED EMBODIMENT

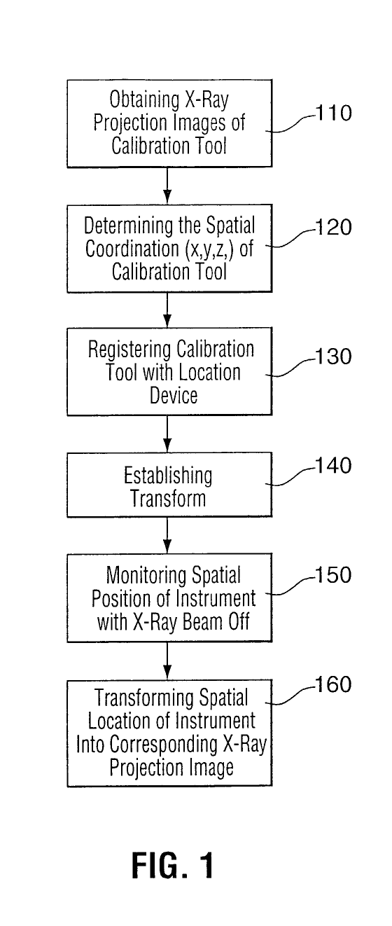

The method of transforming the spatial coordinates of an instrument in the frame of reference of a location device into the corresponding coordinates of an X-ray projection image display will now be described referring to FIG. 1. The method comprises a calibration phase during which X-ray projection images of a calibration tool are acquired (110) while the spatial coordinates (x, y, z) of the calibration tool are determined relative to the X-ray screen and the X-ray source (120) and registered in the frame of reference of a location device (130). The spatial coordinates together with the digitized image information are then treated to establish a transform (140) to convert the spatial coordinates of the instrument into the corresponding X-ray projection image display. The method further comprises an operation mode in which the location device monitors the spatial position of an instrument with the X-ray beam turned off (150). The transform established during the calibration phase is then used to convert the spatial coordinates in the frame of reference of the location device into an X-ray projection image of the instrument (160). Furthermore, the image of the instrument can be simultaneously displayed with the image of an object to be operated on thus enabling an operator to guide the instrument within the object.

By transform it is meant the application of a mathematical operation (or operations) to the spatial coordinates (x, y, z) of an instrument or calibration tool to generate the corresponding coordinates (i, j) of the X-ray projection image. The mathematical operation may involve solving an equation of the type

(i,j)=A(x,y,z) (1)

where A represents a matrix. The values of the elements of the matrix can be obtained during the calibration phase as follows: the X-ray projection image of a calibration tool is acquired while its spatial coordinates are determined and registered within the frame of reference of the location device. Next, a first X-ray projection image is calculated by using pre-determined initial values in matrix A for transforming the spatial coordinates into calculated image coordinates (ical, jcal). The values of the matrix are then iteratively modified until the distance between the coordinates of the real image (ireal, jreal) and the corresponding coordinates of the calculated image is minimized, that is to say, until the difference falls under a predetermined value. By pre-determined initial values it is meant values that will allow the convergence of the iteration. The minimization can be achieved, for example, by using Powell's method described in Computer Journal 7, 155-162, 1964 and U.S. Pat. No. 5,873,822 to Ferre, to find the minimum of a function. This method requires that at least three calibration points be determined. Other methods, known in the art, can also be used to determine the values of the elements of matrix A in the transformation equation that will minimize the distance between the calculated and real image coordinates. Once this calibration is completed, the transformation equation can be applied to calculate the X-ray projection image of any object.

Location systems are well known in the art and may comprise but are not limited to optical and electromagnetic systems. Optical systems include a camera that tracks light signals from the tips of instruments and records the position of the instruments relative to a predefined reference frame. The emission of light from the instrument can either be passive or active. In the active mode the instruments are equipped with a light source that emits light which is detected by the camera. This mode requires that the instruments be wired or be provided with power sources. In the passive mode the instruments usually bears reflectors on which light is shone and reflected to be detected by CCD cameras for example. Such optical location devices typically use infrared light such as the Scout™system marketed by SNS. The efficiency of optical locating devices can be limited by the fact that the instrument must be “visible” to the camera in order to be detected.

Electromagnetic location systems use a transmitter of electromagnetic field located near the instruments which possess a receiver. While these systems overcome the limitation of optical devices, they have the disadvantage of being sensitive to distortion created by ferromagnetic objects.

As will be obvious to persons skilled in the art, both systems may be used as the location device of the method described herein.

Some location devices measure the position of one or more targets on the tool or instrument. By target, it is meant a device enabling the tool or instrument to be “seen” by the location device. For example, the target in the infrared location system described above is the light reflector. The nature of the targets suitable for detection depends on the type of location device as is well known to those skilled in the art. Thus it will be appreciated that location devices, relying on targets to track the position of an instrument, measure the position of the target without measuring the position of the different parts of the instrument. Consequently, in order to register the spatial position of the instrument in the frame of reference of the location device, the position of the target relative to the different parts of the instrument or of the calibration tool should be determined. The parameters defining the relative position of the target on the tool or instrument are stored in the computer to be part of the transform.

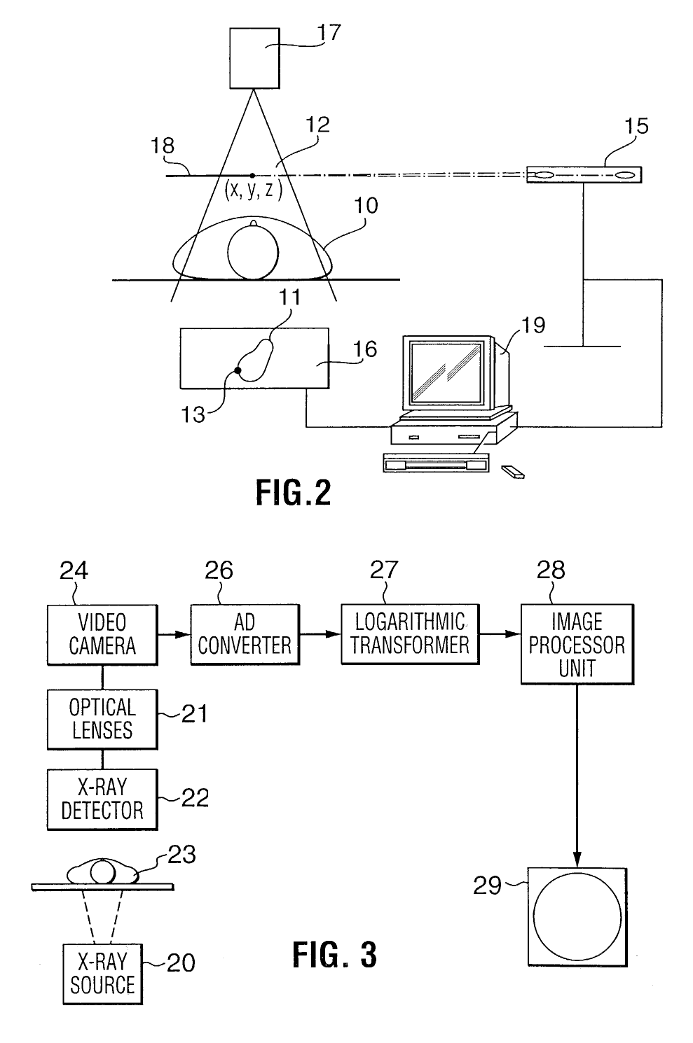

Referring to FIG. 2, the calibration phase will be described. The spatial coordinates (x, y, z) of a substantially radiopaque calibration tool 18 are determined within the X-ray beam 12 relative to an X-ray sensitive screen 16 and an X-ray source 17. By radiopaque calibration tool it is meant a tool exhibiting a high X-ray absorbance. The registration procedure involves placing the calibration tool 18 in the X-ray beam and registering its position in the frame of reference of a location system 15 while acquiring X-ray projection images. A computer 19 is used to digitize the projection images using methods that are well known in the art. The digitized images are then analyzed to determine the location and intensity of the pixels of the projection image of the calibration tool as well as its shape parameters. These parameters are then stored in the computer to be used to establish the transform. By shape parameters, it is meant the geometrical characteristics of the tool. The calibration procedure is repeated at least three times with the calibration tool being displaced each time to a different non-colinear location within the X-ray beam. The data from the location system and from the digitized display of the X-ray projections are then treated to establish the transform used to convert the spatial coordinates obtained with the location system into a display of an instrument on the screen.

In one embodiment, the calibration tool consists of a metal sphere of a known diameter, for example 20 mm, mounted at the end of a thin holder and having a target attached thereto. This sphere, when placed in the X-ray beam, casts a circular shadow on the X-ray sensitive screen. Using image processing techniques well known in the art, the location of the center of the circle and the circle's diameter can be automatically detected and computed. The position of the center on the X-ray screen together with the position of the X-ray source define a line passing through the center of the sphere. Using the X-ray beam projection geometry, which is typically cone shaped, the known distance between the X-ray source and the X-ray sensitive screen and the known diameter of the sphere, the exact position of the sphere along the aforementioned line can be geometrically computed. This provides the 3-D location of the center of the sphere in the frame of reference of the imaging system. The location device simultaneously registers the 3-D location of a target on the sphere in its own coordinate system. Knowing the position of the target relative to the center of the sphere, the spatial coordinates of the different parts of the tools may be determined in the frame of reference of the location device. This calibration process is repeated at least three times to obtain the data that will be used to transform the spatial location of the tool or instrument into the corresponding X-ray projection image.

While the calibration has been described using a sphere as calibration-tool, tools exhibiting other shapes may also be used as would be obvious to one skilled in the art.

It will be appreciated that X-ray images of a calibration tool or of an instrument, other than a sphere, oriented at different angles relative to the X-ray beam but located substantially at the same position may be appreciably different. Furthermore, because the X-ray beam is typically cone-shaped, the surface of the projection image of the calibration tool or of the instrument will be larger than the actual cross-sectional surface of the tool or instrument intercepted by the X-ray beam. In this regard, it will be further appreciated that the surface of the projection will depend on the shape of the beam and the angle of the tool or instrument relative to the beam. Thus it is preferable that the angle of the calibration tool relative to the beam be varied during the calibration phase so as to optimize the transformation. Even more preferably, these angles should correspond to those most likely to be used during the operation mode.

In one embodiment of the instant method the computer generated X-ray projection image of an instrument using the procedure described above, is to be displayed simultaneously with a previously stored image of an object to be operated on. In this case, the calibration may be performed while the object to be operated on, such as the organ of a patient 10 as shown in FIG. 2, is also positioned in the X-ray beam such that the shadow of the object 11 is seen on the X-ray sensitive screen. The calibration tool is then introduced in the X-ray field above the object and X-ray projection images 13 of the calibration tool are acquired simultaneously with images of the object. Once the calibration is completed, the X-ray beam may be turned off and the operating phase, during which the stored image of the object will be simultaneously displayed with the computer generated instrument image, may be entered. Optionally, images of the object to be operated on may be acquired before or after the calibration and stored in a computer for future retrieval.

In the operating mode, procedures such as, but not limited to, surgery can be performed. During the procedure, the location of the instrument is continuously monitored by the location device and processed by the computer. As would be obvious to one skilled in the art, the position of the sensor of the location device will be chosen to permit the detection of the instrument and/or the target located on the instrument. The spatial coordinates of the instrument in the frame of reference of the location device are then transformed to generate an X-ray projection image superimposed on the image of the object that is simultaneously displayed on the screen.

It will also be appreciated that the method can be used for multiple planes X-ray fluoroscopy such as bi-plane fluoroscopy. In this particular case a calibration is performed for each plane of projection. As a result, the image of the instrument can be displayed in three dimensional image reconstructions of the object to be operated on.

As would be obvious to those of skills in the art it is preferable that a new image of the object to be operated on be acquired should it be displaced relative to the X-ray sensitive screen and X-ray source during the procedure so that the proper orientation of the instrument relative to the object be displayed.

To optimize the quality of the computer generated image of the instrument, the calibration is preferably performed with tools similar to the instruments to be used during the operating mode. In particular, the shape and the X-ray absorbing properties should be similar. In a preferred embodiment, the characteristics of the calibration tools and of the instruments to be used in the operating mode are stored in a computer and taken into account in the transform to compensate for any differences between the calibration tools and the instruments.

In a preferred embodiment of this invention, the intensity of X-rays transmitted through an object is recorded on an image intensifier screen which can be a fluorescent screen, although other type of screens can be used and are well known in the art. Images acquired on fluorescent screens are referred to as fluorograms. These screens allow the rapid acquisition of multiple frames. The following is a description of a typical imaging system and is schematically represented in FIG. 3 for explanatory purposes only and is not intended to restrict the scope of the invention. Other arrangements as would be obvious to one skilled in the art are also considered to be within the scope of the invention. The X-ray source 20 generates X-rays upon application of a high voltage. The X-ray detector 22, which is an image intensifier, detects X-rays transmitted through the object 23. The detector 22 also functions to electron-multiply the detected X-rays for conversion into an optical image. It is preferable that the size of the X-ray detecting surface of the X-ray detector 22 can cover that part of the X-rays which are transmitted through the object 23. A TV (video) camera 24 is coupled to the X-ray detector 22 through an optical lens 21 to convert the optical image into an electrical signal. The TV camera 24 is controlled in a well known manner by a TV camera controller which in turn amplifies suitably the electrical output signal of the TV camera 24. The amplified signal is converted into a digital value by an A/D converter 26, and a logarithmic transformer 27 transforms the digital output of the A/D converter 26 into a logarithmic value which represents X-ray absorbance of the object 23. The logarithmic output of the logarithmic transformer 27 is applied to an image processor unit 28 which converts the signal into an image displayed on screen 29. Alternatively, the A/D conversion may be carried out after the logarithmic conversion. Such an imaging system can acquire images at approximately 30 screens/second.

The above method will now be described with reference to image guided surgery. More particularly, the method will be described with reference to image guided surgery using navigation catheters. However, other surgical instruments are also contemplated to be within the scope of the invention.

In one embodiment of the instant method, the calibration tool may be the same as the instrument. For example navigation catheters, which have an integrated navigation system, can be used to register their own spatial coordinates relative to fiducial points while X-ray projection images of the catheters are obtained. The same catheters may then be used in the operating mode.

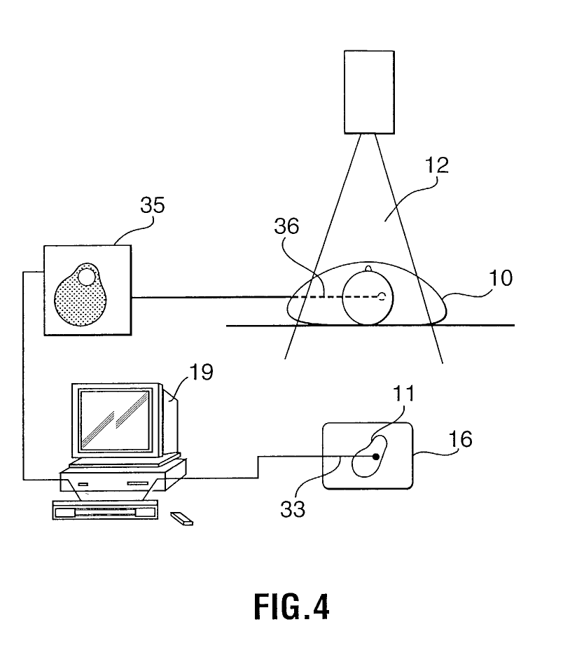

Now referring to FIG. 4, a patient 10 is positioned within the cone-shaped X-ray beam 12 of the fluoroscope such that an X-ray projection image of the patient's organ 11 is seen on the screen 16. In the calibration phase, the integrated navigation system 35, which comprises a navigation catheter 36, is used to establish the coordinates of the catheter relative to fiducial points of the system (not shown) while X-ray image projection 33 of the catheter are obtained. The fiducial points may be the location of a magnetic field generator or devices mounted on other catheters or on the body surface.

Under fluoroscopic guidance and while images are acquired on the X-ray sensitive screen for calibration purposes, the operator moves the catheter within the patient's organ to the extreme borders of the organ. Calibration accuracy is likely to degrade away from the area in which the tip was moved during the calibration phase. Therefore, it is preferable that the calibration be performed while the catheter is at the approximate area in which it will be used during the procedure. The positional data and the X-ray projection images are then processed by a computer according to the method described above i.e. by determining the position of the catheter relative to the X-ray source and the X-ray sensitive screen, registering its position with the location device and establishing a transform. Then the X-ray beam is switched off and the operating mode can be entered.

In a further embodiment, the instant method can be applied to navigate instruments within organs that exhibit motion such as the heart. Both static or “cine” images of the moving organ can be obtained. To obtain static images of the heart, the calibration sequence may be triggered once per heartbeat using the patient's electrocardiogram (ECG) signal. If instead a cine run is desired, the calibration sequence may be sequentially applied during the entire cardiac cycle with the appropriate sequence of images stored for use during the operating phase.

Once the calibration is over, the fluoroscope is switched off while the computer system continues to display the stored fluoroscope image of the heart. This background image may be displayed either as a static image triggered by the patient's ECG or as a dynamic cine loop synchronized with the patient's cardiac cycle through the ECG. The coordinates of the navigation catheter are read from the navigation system either on a continuous basis or in response to a trigger signal generated from the patient's ECG signal. The system then performs a mathematical transform of the navigation coordinates into the fluoroscope coordinates using the previously generated calibration information. The system then generates an image of the navigation catheter and displays it simultaneously with the stored fluoroscope image or sequences of images of the heart.

The resolution of the images obtained using the method of the instant invention can be improved by partly or completely filling the organ with an X-ray contrast agent. The actual concentration of the contrast agent may be adjusted so as to optimize the resolution of the organ and of the instrument and /or calibration tool.

The present invention has been described with regard to preferred embodiments. However, it will be obvious to persons skilled in the art that a number of variations and modifications can be made without departing from the scope of the invention as described herein.

What is claimed is:

1. A method for generating and displaying an X-ray projection image of an instrument located within an object comprising the steps of:
a) registering the spatial coordinates of an X-ray beam imaging system comprising an X-ray source and an X-ray sensitive screen with a location device;
b) recording the spatial location of said instrument using said location device with the X-ray beam switched off;
c) transforming the spatial coordinates of said instrument in the frame of reference of said location device into the coordinates of the corresponding X-ray projection image;
d) displaying said X-ray projection image of said instrument simultaneously with a previously stored X-ray projection image of said object.

2. The method of claim 1 wherein registering the spatial coordinates of said X-ray beam imaging system with said location device comprises the steps of:
a) obtaining, on said X-ray sensitive screen, at least three X-ray projections of a calibration tool, said calibration tool being displaced to a new non-colinear location within the X-ray beam before each projection;
b) determining, for each projection, the spatial coordinates of said calibration tool relative to the X-ray screen and X-ray source;
c) recording, for each projection, the spatial location of the calibration tool relative to said X-ray sensitive screen and said X-ray source with said location device;
d) digitizing, for each projection, the pixels intensity and location on said X-ray sensitive screen and storing them in a computer;
e) establishing a transform to convert the spatial location of said instrument in the frame of reference of said location device into the corresponding coordinates of the corresponding X-ray projection image.

3. The method of claim 2 wherein recording the spatial location of said instrument or calibration tool comprises the steps of:
i) measuring the location of a target on said instrument or calibration tool relative to said X-ray source and said X-ray screen using said location device; and
ii) determining the position of the target relative to the different part of said instrument or calibration tool.

4. The method of claim 3 wherein the calibration tool is a sphere.

5. The method of anyone of claim 5 wherein said X-ray projection image of said object is obtained prior, during or after the registration of the coordinates of the X-ray beam imaging system and stored in a computer to be subsequently retrieved.

6. The method of claim 5 wherein, when said X-ray projection image of said object is obtained during said registration, said object is positioned in the X-ray beam between the calibration tool and the X-ray sensitive screen to acquire X-ray projections of said object simultaneously with X-ray projections of said calibration tool.

7. The method of claim 2 wherein said calibration tool is the same as said instrument.

8. The method of claim 7 wherein a location device is integrated within said instrument.

9. The method of claim 8 wherein said recording of the spatial coordinates is relative to fiducial points.

10. The method of claim 9 wherein said instrument is a navigation catheter.

11. The method of any one of claims 1-10 wherein said object is an organ within a patient.

12. The method of claim 11 wherein the organ is a heart.

13. The method of claim 12 wherein the X-ray projection image of said heart is registered with an electrocardiogram.

14. The method of claim 13 wherein the image of said heart is displayed as a static image triggered by said electrocardiogram.

15. The method of claim 14 wherein multiple images of said heart at different stages of the cardiac cycle are displayed consecutively and in synchrony with said cardiac cycle by triggering the display of each image with the electrocardiogram.

16. The method of claim 2 wherein the registration of the spatial coordinates of the X-ray beam imaging system with said location device is repeated for more than one X-ray projection angle and wherein said transformed coordinates are used to simultaneously display X-ray projection images of the instrument, each said image corresponding to a different X-ray projection angle.

17. A device for transforming the spatial coordinates of an instrument into an X-ray projection image of an instrument according to the method of claim 1, said device comprising an X-ray imaging system, a location device and a computer.
